SEQUENCE LISTING (6 sequences)

SEQ ID NO: 1 - DNA, 633 bp, Artificial Sequence
  GDF11-HSP10 fusion protein
atgaacctgggtctggattgtgacgagcactcttctgaaagccgttgttgccgttaccctctgactgtagacttcgaagcattcggttgggactggatcatcgctccaaaacgctacaaggcgaactactgcagcggtcaatgtgaatacatgtttatgcagaaatacccgcacacccatctggtccagcaggccaacccgcgtggttccgcgggcccgtgttgcacgccgaccaaaatgtctccgattaacatgctgtatttcaatgataaacagcagatcatctatggcaaaattccgggcatggtggttgatcgctgcggctgctccgcgggtcaggcgttccgtaaattcctgccgctgttcgaccgtgttctggttgaacgttctgcggcggaaaccgttaccaaaggtggtatcatgctgccggaaaaatctcagggtaaagttctgcaggcgaccgttgttgcggttggttctggttctaaaggtaaaggtggtgaaatccagccggtttctgttaaagttggtgacaaagttctgctgccggaatacggtggtaccaaagttgttctggacgacaaagactacttcctgttccgtgacggtgacatcctgggtaaatacgttgac

SEQ ID NO: 2 - protein, 211 aa, Artificial Sequence
  GDF11-HSP10 fusion protein
MNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCSAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 3 - DNA, 29 bp, Artificial Sequence
  primer
aaggagatatacatatgaacctgggtctg

SEQ ID NO: 4 - DNA, 23 bp, Artificial Sequence
  primer
ctgacccgcggagcagccgcagc

SEQ ID NO: 5 - DNA, 23 bp, Artificial Sequence
  primer
gctgcggctgctccgcgggtcag

SEQ ID NO: 6 - DNA, 18 bp, Artificial Sequence
  primer
gtgctcgaggtcaacgta

(19) United States
(12) Patent Application Publication
(10) Pub. No.: US 2022/0192958 A1
(43) Pub. Date: Jun. 23, 2022

(54) FUSION PROTEIN COMPRISING GROWTH DIFFERENTIATION FACTOR 11 AND HEAT SHOCK PROTEIN 10 WITH ENHANCED SKIN CELL PROLIFERATION EFFECT AND COSMETIC COMPOSITION FOR ANTI-WRINKLE COMPRISING THE SAME AS EFFECTIVE COMPONENT

(71) Applicants: Sun Kyo LEE, Gyeonggi-do (KR); NEXGEN BIOTECHNOLOGIES, INC., Seoul (KR)

(72) Inventors: Sun Kyo LEE, Gyeonggi-do (KR); Seong Ran LEE, Gyeonggi-do (KR); Han Bong RYU, Seoul (KR); Tae Hyun KIM, Gyeonggi-do (KR)

(21) Appl. No.: 17/599,081

(22) PCT Filed: Aug. 1, 2017
(86) PCT No.: PCT/KR2017/008297

§ 371 Date: Sep. 28, 2021

(30) Foreign Application Priority Data
Apr. 26, 2017 (KR) 10-2017-0053907

(51) Int. Cl. A61K 8/64; C12N 15/62; A61Q 19/08; C07K 14/475; C12N 15/70

(52) U.S. Cl. CPC A61K 8/64; C12N 15/62; C12N 15/70; C07K 14/475; A61Q 19/08

(57) ABSTRACT

A fusion protein according to an embodiment of the present disclosure includes a growth differentiation factor 11 and a heat shock protein 10 with an enhanced skin cell proliferation effect. An anti-wrinkle cosmetic composition according to an embodiment of the present disclosure includes the fusion protein as an effective component, and the cosmetic composition can be advantageously used in future as a material of a functional cosmetic product.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2017/008297, filed Aug. 1, 2017, which claims priority to the benefit of Korean Patent Application No. 10-2017-0053907 filed in the Korean Intellectual Property Office on Apr. 26, 2017, the entire contents of which are incorporated herein by reference.

### BACKGROUND

### 1. Technical Field

The present invention relates to a fusion protein comprising growth differentiation factor 11 and heat shock protein 10 with enhanced skin cell proliferation effect and an anti-wrinkle cosmetic composition comprising the same as effective component.

### 2. Background Art

It is known that various growth factors are involved in growth, proliferation, differentiation of cells, and those growth factors are characterized in that they exhibit their respective functions upon binding to a specific receptor. Due to the short half-life and insufficient stability, it remains difficult to produce those growth factors on a large scale. However, thanks to the progress in genetic recombination techniques, now it becomes possible to overcome the problem.

In 2013, Prof. Amy Wagers of Harvard University (U.S.A) discovered growth differentiation factor 11 (GDF 11) that is closely related to anti-aging. Based on the result indicating that senile rats can recover their youth through parabiosis between them and young rats, she published in 2014 a research paper relating to the anti-aging activity of growth differentiation factor 11. However, researchers of Novartis Biomedical Research Institute opposed the anti-aging effect of growth differentiation factor 11, by focusing on the fact that the amino acid sequence of myostatin, which inhibits muscle growth, is very similar to the amino acid sequence of growth differentiation factor 11. Although the debate is still ongoing, growth differentiation factor 11 is known to be a protein which promotes regeneration and elasticity of skin like fibroblast, collagen, and elastin.

Heat shock protein is one of the proteins that are expressed in cells in response to the exposure to an extreme environment to prevent damages occurring in cell. Most of heat shock proteins have a chaperon function for preventing the functional loss of a protein that is caused by exposure to an extreme environment, and a great amount of energy (i.e., ATP) is required for the process. In skin, high temperature and ultraviolet ray are representative examples of the extreme environment, and, in particular, ultraviolet ray is the direct cause of skin aging. In recent years, studies are made on a heat shock protein which exhibits an ultraviolet ray-shielding effect, and various attempts are also made to use a heat shock protein as a component of a cosmetic composition.

In the present invention, a fusion protein of growth differentiation factor 11 having an excellent skin regeneration effect is developed according to fusion between growth differentiation factor 11 and heat shock protein 10, and a cosmetic composition for regenerating skin and improving skin wrinkle comprising the fusion protein as effective component is also developed.

Meanwhile, in Korean Patent Registration No. 1032271, “Cosmetic composition for skin cell regeneration, method for producing the same, method for skin cell regeneration, and cosmetic composition”, in which the composition comprises vascular endothelial growth factor and fibroblast growth factor, is disclosed, and in Korean Patent Application Publication No. 2016-0084825, “Composition for preventing and alleviating hypersensitive skin response caused by exposure to ultraviolet ray and cosmetic composition using the same”, in which the composition comprises, as an effective component, fermentation extract of onion skin inhibiting Hsp70 is disclosed. However, the fusion protein comprising growth differentiation factor 11 and heat shock protein 10 with enhanced skin cell proliferation effect and an anti-wrinkle cosmetic composition comprising the same as effective component are not disclosed before.

### SUMMARY

The present invention is devised under the circumstances that are described in the above, and the inventors of the present invention prepared a novel fusion protein according to fusion between a gene encoding growth differentiation factor 11 and a gene encoding heat shock protein 10, in which the genes are Escherichia coli (E. coli) codon-optimized. It was also found that, as a result of treating a skin cell line with the fusion protein comprising growth differentiation factor 11 and heat shock protein 10 as prepared, a more excellent cell proliferation effect is obtained from a treatment with the fusion protein compared to a treatment with the individual proteins, and the present invention is completed accordingly.

To solve the problems that are described in the above, the present invention provides a fusion protein comprising growth differentiation factor 11 and heat shock protein 10 with enhanced skin cell proliferation effect in which the fusion protein consists of the amino acid sequence of SEQ ID NO: 2.

The present invention further provides a gene encoding the aforementioned fusion protein.

The present invention further provides a recombinant vector comprising the aforementioned gene.

The present invention further provides a host cell transformed with the aforementioned recombinant vector.

The present invention further provides a method for producing in a host cell a fusion protein comprising growth differentiation factor 11 and heat shock protein 10 including transforming a host cell with the aforementioned recombinant vector.

The present invention further provides a fusion protein comprising growth differentiation factor 11 and heat shock protein 10 produced by the aforementioned method.

The present invention still further provides a cosmetic composition for regenerating skin and improving skin wrinkle comprising, as an effective component, a fusion protein comprising growth differentiation factor 11 and heat shock protein 10 in which the fusion protein consists of the amino acid sequence of SEQ ID NO: 2.

The production method of the present invention in which the production in E. coli is made by using a gene encoding growth differentiation factor 11 and a gene encoding heat shock protein 10, in which the genes are E. coli codon-optimized, enables a simplified production process as the recombinant protein is expressed in form of an inclusion body in E. coli, and the method also enables production of the recombinant protein in large amount. Furthermore, the fusion protein comprising growth differentiation factor 11 and heat shock protein 10 which is produced by the aforementioned method has an excellent skin regeneration effect and an excellent wrinkle-improving effect, and thus it is expected that the fusion protein can be advantageously used as a raw material of novel functional cosmetics.

### BRIEF DESCRIPTION OF THE DRAWINGS

FIG. 1 is a schematic drawing illustrating the process of producing the recombinant plasmid (pET22b::GDF11-HSP10) which contains a gene encoding the fusion protein comprising growth differentiation factor 11 and heat shock protein 10 (i.e., GDF11-HSP10), and transformation of E. coli with the recombinant plasmid.

FIG. 2 shows the result of determining the expression of the fusion protein of the present invention in E. coli and determining the separation and purification of the fusion protein, in which A of FIG. 2 is a result obtained by determining the expression of a fusion protein comprising growth differentiation factor 11 and heat shock protein 10 and the purification process using nickel agarose column, in which the determination was made by electrophoresis using a SDS-PAGE gel, and B of FIG. 2 is a result obtained by electrophoresis of GDF11-HSP10 fusion protein which has been finally separated and purified, in which M represents a size marker; 1 represents a sample which has been solubilized from the inclusion body after expression induction; 2 represents a flow through not adhered to the column; 3 represents a column washing sample; and 4 and 5 represent an elution sample.

FIG. 3 shows the result of determining the cell proliferation of dermal fibroblast cells after treating the cells only with growth differentiation factor 11 (GDF11) or heat shock protein 10 (HSP10), or with a fusion protein comprising growth differentiation factor 11 and heat shock protein 10 (GDF11-HSP10), in which the determination was made based on crystal violet staining after the treatment.

### DETAILED DESCRIPTION

To achieve the object of the present invention, the present invention provides a fusion protein comprising growth differentiation factor 11 and heat shock protein 10 with enhanced skin cell proliferation effect in which the fusion protein consists of the amino acid sequence of SEQ ID NO: 2.

A protein having the amino acid sequence represented by SEQ ID NO: 2 and also functional equivalents of the protein fall within the scope of the fusion protein comprising growth differentiation factor 11 and heat shock protein 10 according to the present invention. The term “functional equivalents” indicates a protein having, as a result of addition, substitution, or deletion of an amino acid, at least 70%, preferably at least 80%, more preferably at least 90%, and even more preferably at least 95% sequence homology with the amino acid sequence represented by SEQ ID NO: 2, and it indicates a protein exhibiting substantially the same activity as the protein represented by SEQ ID NO: 2. The expression “substantially the same activity” means a skin regeneration and wrinkle-improving activity. Also included in the present invention are fragments, derivatives, and analogues of the fusion protein comprising growth differentiation factor 11 and heat shock protein. The terms “fragments”, “derivatives”, and “analogues” that are described in the present specification indicate a polypeptide with the substantially same biological function or activity as the fusion protein comprising growth differentiation factor 11 and heat shock protein 10 of the present invention.

The fusion protein comprising growth differentiation factor 11 and heat shock protein 10 of the present invention preferably consists of the amino acid sequence of SEQ ID NO: 2, and the fusion protein is a novel protein that is produced by fusion between the growth differentiation factor 11 consisting of the 2^{nd} to the 110^{th} amino acids of the amino acid sequence of SEQ ID NO: 2 and heat shock protein 10 consisting of the 111^{th} to the 211^{th} amino acids of the amino acid sequence of SEQ ID NO: 2.

The present invention further provides a gene encoding the fusion protein comprising growth differentiation factor 11 and heat shock protein 10 which has an enhanced skin cell regeneration effect. The gene may consist of the E. coli codon-optimized nucleotide sequence of SEQ ID NO: 1, but it is not limited thereto.

This gene encoding the fusion protein comprising growth differentiation factor 11 and heat shock protein 10 with enhanced skin cell regeneration effect of the present invention may include the nucleotide sequence of SEQ ID NO: 1. Furthermore, homologues of the nucleotide sequence are also within the scope of the present invention. Specifically, the above described gene may comprise a nucleotide sequence which has preferably at least 70%, more preferably at least 80%, still more preferably at least 90%, and most preferably at least 95% homology with the nucleotide sequence selected from a group consisting of nucleotide sequences of SEQ ID NO: 1. The “sequence homology %” for a certain polynucleotide is identified by comparing a comparative region with two sequences that are optimally aligned. In this regard, a part of the polynucleotide in comparative region may comprise an addition or a deletion (i.e., a gap) compared to a reference sequence (without any addition or deletion) relative to the optimized alignment of the two sequences.

“Codon-optimized” means a modification of codon of a polynucleotide encoding a protein with a codon that is used first than others in a specific organism such that the coded protein can be more efficiently expressed therein. Because most amino acids are described by several codons that are referred to as “synonym” or “synonymous codon”, genetic codes have degeneracy. However, codon usage by a specific organism is not random, and it is rather biased to specific codon triplets. Such codon usage bias may be even higher in relation with a certain gene, a gene with common function or ancestor origin, protein expressed at high level vs. proteins with low copy number, or a group protein coding region of a genome of an organism. The nucleotide sequence of SEQ ID NO: 1 of the present invention is a sequence which has been optimized to E. coli codon such that the gene encoding the fusion protein comprising growth differentiation factor 11 and heat shock protein 10 can be expressed in E. coli.

The present invention further provides a recombinant vector comprising the aforementioned gene, and a host cell transformed with the recombinant vector.

The term “recombinant” indicates a cell which replicates a heterogeneous nucleotide or expresses said nucleotide, or a peptide, a heterogeneous peptide, or a protein encoded by a heterogeneous nucleotide. Recombinant cell can express a gene or a gene fragment in the form of a sense or antisense, which are not found in natural state of cell. In addition, a recombinant cell can express a gene that is found in natural state, provided that said gene is modified and re-introduced into the cell by an artificial means.

According to the present invention, the gene encoding a fusion protein comprising growth differentiation factor 11 and heat shock protein 10 can be inserted to a recombinant expression vector. The term “recombinant expression vector” means bacteria plasmid, phage, yeast plasmid, plant cell virus, mammalian cell virus, or other vector. In general, any plasmid and vector can be used if it can replicate and be stabilized in a host. Important characteristics of the expression vector include that it comprises a replication origin, a promoter, a marker gene, and a translation control element.

The expression vector comprising the gene sequence encoding a fusion protein comprising growth differentiation factor 11 and heat shock protein 10 and an appropriate signal for regulating transcription/translation can be constructed according to a method that is well known to a skilled person in the art. The method includes an in vitro recombinant DNA technique, a DNA synthesis technique, and an in vivo recombinant technique. For inducing mRNA synthesis, the DNA sequence can be effectively linked to a suitable promoter present in the expression vector. In addition, the expression vector may comprise a ribosome binding site as a translation initiation site and a transcription terminator.

The recombinant vector according to one embodiment of the present invention is prepared by in-frame fusion of a synthesized gene encoding the fusion protein comprising growth differentiation factor 11 and heat shock protein 10 (i.e., SEQ ID NO: 1) at 5′ terminal (NdeI restriction enzyme site) and 3′ terminal (XhoI restriction enzyme site) in pET22b vector, and it may be a recombinant vector characterized in that it can produce the fusion protein comprising growth differentiation factor 11 and heat shock protein 10 based on effective expression of the aforementioned gene with an aid of lac promoter (lac promoter) and lad repressor (lad repressor), but it is not limited thereto.

As a host cell having an ability of having stable and continuous cloning and expression of the vector of the present invention in a prokaryotic cell, any host cell known in the pertinent art can be used. Examples of the prokaryotic cells include, Bacillus sp. strain including E. coli Rosetta, E. coli JM109, E. coli BL21, E. coli RR1, E. coli LE392, E. coli B, E. coli×1776, E. coli W3110, Bacillus subtillus, Bacillus thuringiensis and the like, and intestinal bacteria and strains including Salmonella typhimurium, Serratia marcescens and various Pseudomonas sp. etc.

Furthermore, when a eukaryotic cell is transformed with the vector of the present invention, yeast (Saccharomyce cerevisiae), an insect cell, a human cell (for example, CHO (Chinese hamster ovary) cell line, W138, BHK, COS-7, HEK 293, HepG2, 3T3, RIN, and MDCK cell line), a plant cell, and the like can be used as a host cell.

The transformed host cell of the present invention may be E. coli Rosetta2 (DE3) pLysS cell line, but it is not limited thereto.

When a host cell is a prokaryotic cell, delivery of the vector of the present invention into a host cell can be carried out by CaCl₂ method, Hanahan's method (Hanahan, D., J. Mol. Biol., 166:557-580 (1983)) or an electroporation method, and the like. In addition, when a host cell is a eukaryotic cell, the vector can be introduced to a host cell by a microinjection method, calcium phosphate precipitation method, an electroporation method, a liposome-mediated transfection method, DEAE-dextran treatment method, or a gene bombardment method, and the like.

The present invention further provides a method for producing in a host cell a fusion protein comprising growth differentiation factor 11 and heat shock protein 10 including transforming a host cell with the aforementioned recombinant vector to overexpress a gene encoding the fusion protein comprising growth differentiation factor 11 and heat shock protein 10.

In the method according to one embodiment of the present invention, the host cell may be preferably E. coli, and more preferably E. coli Rosetta2 (DE3) pLysS cell line, but it is not limited thereto.

The present invention further provides a fusion protein comprising growth differentiation factor 11 and heat shock protein that is produced by the aforementioned method.

The present invention still further provides a cosmetic composition for regenerating skin and improving skin wrinkle comprising, as an effective component, a fusion protein comprising growth differentiation factor 11 and heat shock protein 10 in which the fusion protein consists of the amino acid sequence of SEQ ID NO: 2.

In the cosmetic composition according to one embodiment of the present invention, content of the fusion protein comprising growth differentiation factor 11 and heat shock protein 10 may be 0.000001 to 0.00002% by weight relative to the total weight of the cosmetic composition, but it is not limited thereto.

In the cosmetic composition of the present invention, components that are typically used for a cosmetic composition are included in addition to the effective component described above. Examples thereof include a lipid material, an organic solvent, a dissolution agent, a condensation agent, a gelling agent, a softening agent, an anti-oxidant, a suspension agent, a stabilizer, a foaming agent, an aroma, a surface active agent, water, an ionic or non-ionic emulsifier, a filler, a metal ion sequestering agent, a chelating agent, a preservative, vitamin, a blocking agent, a moisturizing agent, essential oil, a dye, a pigment, a hydrophilic or lipophilic activating agent, a common auxiliary agent such as lipid vesicle, and a carrier.

The composition of the present invention can be prepared in any formulation which is generally prepared in the pertinent art. For example, the composition may be formulated into a solution, a suspension, an emulsion, a paste, a gel, a crème, a lotion, a powder, an oil, a powder foundation, an emulsion foundation, a wax foundation, a spray, or the like, but not limited thereto. More specifically, the composition may be formulated into a skin, a skin softener, a skin toner, an astringent, a lotion, a milk lotion, a moisture lotion, a nutrition lotion, a massage crème, a nutrition crème, an eye crème, a moisture crème, a hand crème, an essence, a nutrition essence, a pack, a cleansing foam, a cleansing water, a cleansing lotion, a cleansing crème, a body lotion, a body cleanser, a soap, a powder, or the like.

In a case in which the cosmetic composition of the present invention has a formulation type of paste, crème, or gel, it is possible to use, as a carrier component, animal oil, plant oil, wax, paraffin, starch, tragacanth, cellulose derivatives, polyethylene glycol, silicone, bentonite, silica, talc, or zinc oxide.

In a case in which the cosmetic composition of the present invention has a formulation type of powder or spray, it is possible to use, as a carrier component, lactose, talc, silica, aluminum hydroxide, calcium silicate, or polyamide powder, when it is spray, in particular, a propellant such as chlorofluoro hydrocarbon, propane/butane, or dimethyl ether may be additionally contained.

In a case in which the cosmetic composition of the present invention has a formulation type of solution or emulsion, a solvent, a dissolution agent, or an emulsifier is used as a carrier component, and examples thereof include water, ethanol, isopropyl alcohol, ethyl carbonate, ethyl acetate, benzyl alcohol, benzyl benzoate, propylene glycol, 1,3-butyl glycol oil, glycerol aliphatic ester, polyethylene glycol, and fatty acid ester of sorbitan.

In a case in which the cosmetic composition of the present invention has a formulation type of suspension, it is possible to use, as a carrier component, a liquid phase diluent such as water, ethanol, or propylene glycol, a suspension agent such as ethoxylated isostearyl alcohol, polyoxyethylene sorbitol ester, or polyoxyethylene sorbitan ester, microcrystalline cellulose, aluminum metahydroxide, bentonite, agar, or tragacanth.

Hereinbelow, the present invention is explained in greater detail in view of the Examples. However, it is evident that the following Examples are given only for exemplification of the present invention and by no means the present invention is limited to the following Examples.

### EXAMPLES

### Example 1. Preparation of Recombinant Expression Vector and Transformed Recombinant Microorganism for Producing Fusion Protein Comprising Growth Differentiation Factor 11 and Heat Shock Protein 10

The optimized gene encoding the fusion protein comprising growth differentiation factor 11 and heat shock protein 10 of the present invention, recombinant expression vector, and transformed recombinant microorganism were produced according to the following methods.

By using as a template a gene encoding growth differentiation factor 11 and a gene encoding heat shock protein 10, which is used as a partner protein, a gene (SEQ ID NO: 1) fragment encoding a fusion protein comprising growth differentiation factor 11 and heat shock protein 10, which consists of 211 amino acids and is optimized such that it can be expressed in a host microorganism, was synthetically prepared.

To synthesize a gene encoding the fusion protein (SEQ ID NO: 2) comprising growth differentiation factor 11 and heat shock protein 10 in which heat shock protein 10 is linked to the C-terminus of growth differentiation factor 11, 330 nucleotides (i.e., 1^{st} to 330^{th} nucleotide sequence of SEQ ID NO: 1) encoding E. coli-optimized growth differentiation factor 11 was synthesized by using forward primer 1 (5′-AAGGAGATATACATATGAACCTGGGTCTG-3′; SEQ ID NO: 3) and reverse primer 1 (5′-CTGACCCGCGGAGCAGCCGCAGC-3; SEQ ID NO: 4). In addition, 303 nucleotides (i.e., 331^{st} to 633^{rd} nucleotide sequence of SEQ ID NO: 1) encoding E. coli-optimized heat shock protein 10 was synthesized by using forward primer 2 (5′-GCTGCGGCTGCTCCGCGGGTCAG-3; SEQ ID NO: 5) and reverse primer 2 (5′-GTGCTCGAGGTCAACGTA-3; SEQ ID NO: 6). By having the gene encoding growth differentiation factor 11 protein and the gene encoding heat shock protein 10, which have been synthesized according to the above method, as a template and using forward primer 1 (SEQ ID NO: 3) and reverse primer 2 (SEQ ID NO: 6), a gene consisting of 633 nucleotides which encodes the fusion protein having heat shock protein 10 linked to the C-terminus of growth differentiation factor 11 was finally synthesized via polymerase chain reaction (PCR).

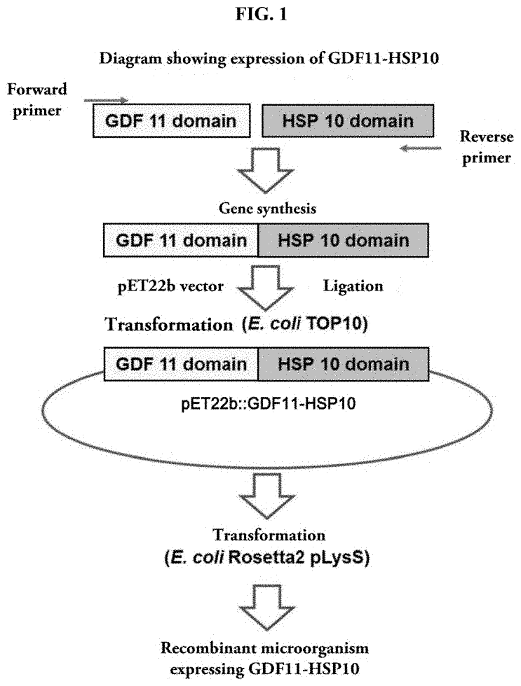

According to digestion of the above gene fragment and recombinant plasmid with the same restriction enzymes (5′ terminus NdeI and 3′ terminus XhoI) followed by insertion, the recombinant plasmid (pET22b::GDH10) shown in FIG. 1 was prepared. By transforming E. coli TOP10 with the prepared recombinant plasmid, the gene construct was obtained in large amount from the host microorganism.

After that, E. coli Rosetta2 (DE3) pLysS (Novagen, Germany) was transformed with the prepared recombinant plasmid so that a recombinant microorganism for producing the fusion protein comprising growth differentiation factor 11 and heat shock protein 10 was prepared.

### Example 2. Induced Expression, Separation, and Purification of Fusion Protein Comprising Growth Differentiation Factor 11 and Heat Shock Protein 10

E. coli Rosetta2 (DE3) pLysS prepared in Example 1 was cultured by using 1 l LB medium (10% tryptophan, 10% sodium chloride, and 5% yeast extract) or BSS medium (1% tryptophan, 0.5% yeast extract, 1% glucose, and 0.1% HEPES (pH 7.0), Nexgen Biotechnologies, Inc.) till to have OD₆₀₀=0.6 to 0.8 for batch culture, or OD₆₀₀=15 to 20 for continuous culture which uses a 20 l fermenter. After that, by adding 1 to 5 mM IPTG or 2% lactose (each at final concentration) to the cell culture medium, gene expression of the recombinant E. coli was induced. After inducing the gene expression, the cells were additionally cultured for 3 to 4 hours, and then collected by centrifuge. The resulting cells were completely suspended in phosphate buffered saline (8 g sodium chloride, 0.2 g potassium chloride, 1.44 g sodium hydrogen phosphate (Na₂HPO₄), and 0.24 g potassium dihydrogen phosphate (KH₂PO₄)/l, pH 7.4), and then disrupted by using an ultrasonic homogenizer so as to separate a solution containing the intracellular proteins.

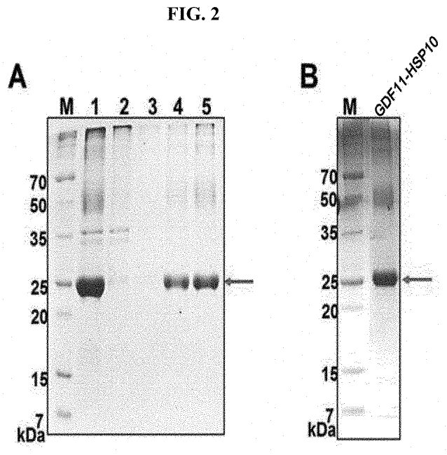

By using thus-separated solutions as a sample, protein expression was determined by 15% SDS-polyacrylamide gel electrophoresis. As a result, it was found that the fusion protein comprising growth differentiation factor 11 and heat shock protein 10 is expressed in a crude lysate of cells which have been induced by IPTG or lactose to undergo the expression (A of FIG. 2).

In order to separate and purify the fusion protein comprising growth differentiation factor 11 and heat shock protein 10 of which expression has been confirmed, the inclusion body was solubilized with a solubilizing buffer solution (5 M urea, pH 11), and then subjected to a refolding process by ultrafine filtration (0.45 μm fine filtration membrane and 1 K ultrafine filtration membrane). By using a buffer solution for storage (PBS), the fusion protein comprising growth differentiation factor 11 and heat shock protein 10 was finally separated.

For having complete purification of the above fusion protein, the separated fusion protein was passed through a nickel-agarose column at a rate of 1 to 3 ml/minute. Subsequently, the column was washed several times with a binding buffer solution, and an imidazole solution (pH 7.4) at a concentration of 50, 100, or 250 mM was applied to the column to fractionate and elute the fusion protein comprising growth differentiation factor 11 and heat shock protein 10, in which each fraction is eluted in an amount of 1 ml. Then, the imidazole in the buffer was removed by using 10 mM potassium phosphate solution so that the fusion protein was finally purified in pure state. To examine the result, 15% SDS-polyacrylamide gel electrophoresis was carried out. As a result, the finally purified fusion protein was found near the region having the expected size (about 24 to 25 kDa including His tag) (B of FIG. 2).

### Example 3. Activity Measurement of Fusion Protein Comprising Growth Differentiation Factor 11 and Heat Shock Protein 10: Dermal Fibroblast Cell Proliferation Effect

After selecting samples from which the presence of the fusion protein comprising growth differentiation factor 11 and heat shock protein 10 has been confirmed as the fusion protein is separated and purified in Example 2, activity of the fusion protein was measured.

Dermal fibroblast cells (Human Dermal Fibroblasts adult, HDFa cell) were cultured, and then treated with growth differentiation factor 11 (GDF11) or heat shock protein 10 (HSP10), or with the fusion protein comprising growth differentiation factor 11 and heat shock protein 10, each at a concentration of 0, 0.02 ppm, or 0.2 ppm, followed by culture for 3 days at 37° C. After that, proliferation of the dermal fibroblast cells was examined by crystal violet staining.

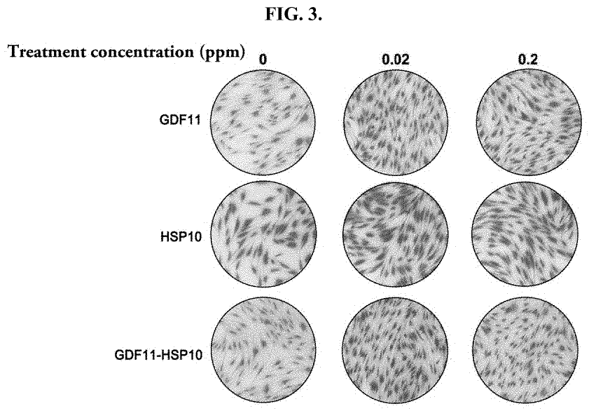

As a result, it was found that a more excellent dermal fibroblast proliferation effect is obtained as the concentration of the fusion protein comprising growth differentiation factor 11 and heat shock protein 10 increases (FIG. 3). It was also observed that the cell proliferation effect exhibited by the fusion protein comprising growth differentiation factor 11 and heat shock protein 10 (i.e., GDH10) is similar to the effect of a group treated with single protein (i.e., active domain of GDF11 or HSP10). In this regard, instead of being a full-length protein, each of GDF11 and HSP10 in the fusion protein corresponds to the separate active domain that has been used for producing the GDH10 fusion protein, and, when the treatment is carried out with each single protein or the fusion protein at same concentration (e.g., 0.02 ppm), mole number of the fusion protein would be about ½ of the mole number of each single protein (i.e., active domain of GDF11 or HSP10). As such, if there is a similar dermal fibroblast proliferation effect at the same concentration, the fusion protein has a dermal fibroblast proliferation effect that is about 2 times higher than the proliferation effect of each of GDF11 and HSP10. As it can be seen from FIG. 3, since the dermal fibroblast proliferation effect obtained by a treatment with the fusion protein comprising growth differentiation factor 11 and heat shock protein 10 is similar to the effect of GDF11 or HSP10 only, it is found that the dermal fibroblast proliferation effect of the fusion protein is at least 2 times higher than each of GDF11 and HSP10. Based on this result, it was found that the fusion protein comprising growth differentiation factor 11 and heat shock protein 10 according to the present invention has an excellent skin cell proliferation effect.

A sequence listing electronically submitted with the present application on Sep. 28, 2021 as an ASCII text file named 20210928_Q61421GR13_TU_SEQ, created on Sep. 16, 2021 and having a size of 4,000 bytes, is incorporated herein by reference in its entirety.

1. A fusion protein having the amino acid sequence of SEQ ID NO: 2.

2. A gene encoding the fusion protein of claim 1.

3. The gene according to claim 2, wherein the gene has the nucleotide sequence of SEQ ID NO: 1.

4. A recombinant vector comprising the gene of claim 2.

5. A host cell transformed with the recombinant vector of claim 4.

6. A method for producing the fusion protein in a host cell, the method comprising transforming the host cell with the recombinant vector of claim 4 to overexpress the gene.

7. The method according to claim 6, wherein the host cell is E. coli.

8. A composition comprising, as an effective component, a fusion protein having the amino acid sequence of SEQ ID NO: 2.

9. A recombinant vector comprising the gene of claim 3.

10. A host cell transformed with the recombinant vector of claim 9.

11. A method for producing the fusion protein in a host cell, the method comprising transforming the host cell with the recombinant vector of claim 9 to overexpress the gene.

12. The method according to claim 11, wherein the host cell is E. coli.

13. A method for regenerating skin and improving skin wrinkle of a subject, the method comprising applying the composition of claim 8 to a skin of the subject.
